(19) United States
(12) Patent Application Publication
(10) Pub. No.: US 2011/0091420 A1
(43) Pub. Date: Apr. 21, 2011

(54) Injectable Sustained-Release Pharmaceutical Formulation and the Preparation Method Thereof

(75) Inventors: Keliang Liu, Beijing (CN); Dongqin Quan, Beijing (CN); Yuanjun Liang, Beijing (CN); Qingbin Meng, Beijing (CN); Chenhong Wang, Beijing (CN); Junlin He, Beijing (CN); Qiyan Jia, Beijing (CN); Sicheng Li, Chengdu (CN)

(73) Assignees: Institute of Pharmacology and Toxicology, Academy of Military Medical Sciences, People Liberation Ar, Beijing (CN); Chengdu Yiping Pharmaceutical Science Development Co., Ltd., Chengdu (Sichuan) (CN)

(21) Appl. No.: 12/933,669

(22) PCT Filed: Mar. 20, 2009
(86) PCT No.: PCT/CN09/70913

§ 371 Date: Dec. 13, 2010

(30) Foreign Application Priority Data
Mar. 20, 2008 (CN) PCT/CN2008/000551

(51) Int. Cl. A61K 38/21; A61K 31/415; A61K 31/44; A61K 48/00; A61K 38/07; A61K 38/31; A61K 38/22; A61K 38/28; A61K 38/38

(52) U.S. Cl. 424/85.4; 514/397; 514/282; 514/44 R; 514/21.9; 514/11.1; 514/11.9; 514/5.9; 514/15.2; 514/6.9; 514/9.7

(57) ABSTRACT

Disclosed are an injectable sustained-release pharmaceutical formulation and a process for preparing the same. In some embodiments, the formulation comprises an active ingredient in a therapeutically effective amount, an amphipathic molecule, an organic acid and/or a salt thereof which is hardly soluble in water, and an oily solvent. The injectable sustained-release pharmaceutical formulation provides a good sustained-release effect for various active ingredients, in particular peptides, proteins, nucleic acids and saccharides.

### CROSS REFERENCE OF RELATED APPLICATIONS

The present application claims the benefit of the international application PCT/CN2008/000551, titled “Injectable Sustained-Release Formulation and Process for Preparation thereof”, which was filed on Mar. 20, 2008, and of which all the contents are incorporated herein by reference in its entirety.

### TECHNICAL FIELD

The present application relates to a sustained-release pharmaceutical composition, in particular to a sustained-release composition of hydrophilic biological drugs such as peptides, proteins, nucleic acids, saccharides and the like. The present application further relates to an injectable sustained-release pharmaceutical formulation prepared from the sustained-release pharmaceutical composition and to a process for preparing the injectable sustained-release pharmaceutical formulation.

### TECHNICAL BACKGROUND

With the fast development of biological technology, biological drugs such as peptides, proteins, nucleic acids, saccharides and the like are becoming a group of important therapeutic agents.

Although the efficacy of biological drugs has been demonstrated by clinical studies, comparing with small-molecule drugs, biological drugs suffer from lower stabilities and are more liable to deactivation. In addition, most of biological drugs belong to hydrophilic large-molecule materials with low lipid/water partition coefficient and are therefore difficult to be taken in by lipophilic membranes, which results in that biological drugs are difficult to pass biological barriers. Therefore, the oral bio-availabilities of biological drugs are normally low.

Accordingly, for biological drugs, a good route of administration is through parental administration, e.g. through injection. However, for patients who need to maintain a certain level of drug concentration in blood, this kind of administration shall be performed repeatedly. Therefore, a sustained-release formulation of biological drugs was developed recently in order to improve the rationality and efficiency of the administration.

Suspensions or solutions prepared by dissolving drugs in oily solvents would have sustained-release effects. However, when drugs with high water-solubilities, e.g. biological drugs, are suspended or partially dissolved in oils, the drugs are liable to entering into the water phase when reaching the oil/water interface. Therefore, for biological drugs which have high water-solubilities or high polarities, sustained-release effects are difficult to be achieved by simply utilizing oily suspensions.

In certain therapeutic fields, liposome has been successfully uses as the vehicle for releasing biological drugs. However, as a sustained-release system; liposome still has some issue to solve, for example, under certain conditions, the sustained-release effect is not satisfactory, encapsulating ratio is low, the physical and chemical stability is poor, etc.

Although a notable progress has been made to sustained-release formulations of drugs such as peptides, proteins, nucleic acids and saccharides, and some injectable sustained-release formulations have been successfully marketed, this kind of formulations in the art are still not satisfactory due to their complex manufacturing process and rigid operational requirements.

Accordingly, various new sustained-release pharmaceutical formulations are still needed to meet different therapeutic requirements.

### SUMMARY

In one aspect, the present application relates to a sustained-release pharmaceutical composition, comprising a therapeutically effective amount of an active ingredient, an amphipathic molecule, an organic acid and/or a salt thereof which is hardly soluble in water, and an oily solvent.

In another aspect, the present application relates to an injectable sustained-release pharmaceutical formulation prepared from the sustained-release pharmaceutical composition disclosed herein.

In a further aspect, the present application provides a process for preparing a injectable sustained-release pharmaceutical formulation, comprising:

(1) dissolving or suspending an active ingredient into an aqueous solvent;

(2) dissolving or suspending an amphipathic molecule and an organic acid and/or a salt thereof which is hardly soluble in water into an organic solvent;

(3) dispersing the aqueous mixture of the active ingredient obtained in step (1) into the organic mixture obtained in step (2);

(4) removing the organic solvent from the mixture obtained in step (3);

(5) drying the products obtained in step (4) to form a solid; and

(6) dissolving or suspending the solid obtained in step (5) into an oily solvent.

In a further aspect, the present application provides an injectable sustained-release pharmaceutical formulation, which comprises an active ingredient in a therapeutically effective amount, an amphipathic molecule, an organic acid and/or a salt thereof which is hardly soluble in water, and an oily solvent, the injectable sustained-release pharmaceutical formulation is prepared by the steps of:

(1) dissolving or suspending the active ingredient into an aqueous solvent;

(2) dissolving or suspending the amphipathic molecule and the organic acid and/or a salt thereof which is hardly soluble in water into an organic solvent;

(3) dispersing the aqueous mixture of the active ingredient obtained in step (1) into the organic mixture obtained in step (2);

(4) removing the organic solvent from the mixture obtained in step (3);

(5) drying the products obtained in step (4) to form a solid; and

(6) dissolving or suspending the solid obtained in step (5) into the oily solvent.

In a further aspect, the present application provides a process for treating a subject, comprising administrating to the subject a therapeutically effective amount of a pharmaceutical composition or a sustained-release pharmaceutical formulation of the present application.

The sustained-release pharmaceutical formulation of the present application provides a good sustained-release effect for hydrophilic biological drugs, in particular peptides, proteins, nucleic acids and saccharides.

### DETAILED DESCRIPTION OF EMBODIMENTS

In one aspect, the present application relates to a sustained-release pharmaceutical composition, comprising an active ingredient in a therapeutically effective amount, an amphipathic molecule, and an organic acid and/or a salt thereof which is hardly soluble in water

The active ingredient may be used in the composition of the present application is a hydrophilic drug, including but not limited to:

peptides and proteins, for example, pituitary polypeptides such as adrenal cortical hormone, gastrin, vasopressin, oxytocin, melanoma stimulating hormone, and the like; gastrointestinal peptides such as secretin, gastrin, cholecystokinin, gastrone, vasoactive intestinal peptide, pancreatic polypeptide, neurotensin, frog skin peptide, and the like; hypothalamic peptides such as thyrotropin releasing hormone, gonadotropin releasing hormone, somatostatin, growth hormone releasing hormone, MSH cytokine inhibiting hormone, and the like; brain peptides such as enkephalin, neoendorphine, endorphin, memory peptide, and the like; kinins such as angiotensins I, II, III, and the like; glutathione; calcitonin; sleep-inducing peptides; pineal peptides; solcoseryl; thymosin; thymopentin; octreotide; exenatide; pramlintide; fibrous proteins; fibrinogens; gastric mucin; gelatin; gelatin sponge; protamines; endostatins; exendin; parotin; hirudin; hepatocyte growth factors; leuprorelin; triptorelin; nafarelin; goserelin; buserelin; bovine serum albumins; insulin; erythropoietin (EPO); tumor necrosis factors; vaccines; auxins; glucagons; serum albumins; gamma-globulins; trypsin inhibitors; erythropoietins; interferons; interleukins; colony-stimulating factors (GM-CSFs); luteinizing hormones, phytohemagglutinin, trichosanthin, plant toxic proteins; antibodies, and the like;

nucleic acids, for example, DNA fragments such as DNA fragment comprising 33 base pair, chemically modified DNA fragments such as thio-DNA fragments, RNA fragments, chemically modified RNA fragments, polyinosinic acid, mecapto polycytidylic acid, cAMP, CTP, CDP-choline, GMP, IMP, AMP, inosine. UTP, NAD, NADP, 2-methylmercapto furan inosinic acid, bisformyl cAMP, 6-mercaptopurine, 6-mercaptopurinenucleoside, 6-thiopurine, 5-fluorouracil, furan fluorouracil, from organic bases include but not limited to salts of isopropylamine, diethylamine, 1,2-diaminoethane, ethanolamine, diethanolamine, trimethylamine, dicylcohexylamine, choline, caffeine, and the like.

In some preferred embodiments, the active ingredient in the composition of the present application may be leuprorelin acetate, or triptorelin acetate.

Other pharmaceutically acceptable derivatives of the active ingredient are those well-known to a person skilled in the art, including but not limited to prodrugs thereof.

“Prodrug” refers to a compound which can be converted to an active ingredient through solvent decomposition under physiological conditions. Accordingly, the term “prodrug” refers to a pharmaceutically acceptable metabolic precursor of the active ingredient in the composition of the present application. Examples of the prodrug include but not limited, to acetate, formate, benzoate, phosphate, sulfonates derivatives of the alcohol functionality; and ester or amide derivatives of the carboxylic acid functionality, of the active ingredient in the composition of the present application.

The amount of the active ingredient comprised in the composition of the present application is based on achieving a therapeutically effective amount.

“Therapeutically effective amount” refers to the amount of the active ingredient in the composition of the present application, which is sufficient to achieve treatment/prevention of a disease or condition to be treated/prevented in a mammal, especially human being, when it is administered thereto. The amount of the active ingredient in the composition of the present application constituting a “therapeutically effective amount” may vary according to the type of the active ingredient, the condition and the severity thereof, and the physical conditions of the subject such as age, weight and the like, and may conventionally determined by a person with ordinary skill in the art according to their own knowledge and the disclosure of the present application.

The active ingredient may be a single drug, or a combination of one or more pharmaceutically compatible drugs.

The amount of the active ingredient in the composition of the present application is normally from about 0.0001% to about 50% based on the total amount of the composition (weight percentage, w/w). In some embodiments, the amount of the active ingredient in the 2-deoxynucleoside, cytarabine hydrochloride, antiviral enzyme plasmid gene, and the like;

saccharides, and non-peptide non-nucleic acid organic drugs, for example, polysaccharide drugs such as heparin, pilose antler polysaccharides, polysaccharide from stichopus japonicus, chitosan, dextran, lentinan, tremella polysaccharide, pachymaran. ganoderma lucidum polysaccharides, and the like; chemically synthesized drugs such as naltrexone hydrochloride, morphine hydrochloride, mitoxantrone hydrochloride, cortisone acetate, and the like.

In some preferred embodiment's, the active ingredient in the composition of the present application may include peptides and proteins. In some more preferred embodiments, the active ingredient in the composition of the present application may be selected from the group consisting of thymopentin, bovine serum albumins, exenatide, pramlintide, somatostatin, ω-interferons, octreotide, salmon calcitonin, and insulin.

In some preferred embodiments, the active ingredient in the composition of the present application may be nucleic acids. In some more preferred embodiments, the active ingredient in the composition of the present application may be selected from oligonucleotide.

In some preferred embodiments, the active ingredient in the composition of the present application may be saccharides and non-peptide non-nucleic acid organic drugs. In some more preferred embodiments, the active ingredient in the composition of the present application may be selected from naltrexone hydrochloride.

In some embodiments of the present application, the active ingredient may be pharmaceutically acceptable salts or other derivatives thereof.

The pharmaceutically acceptable salts of the active ingredient are those well-known to a person skilled in the art, including acid addition salts and base addition salts. Exemplary acids include inorganic salts such as hydrochloric acid, sulfuric acid, phosphoric acid, hydrobromic acid, boric acid, and the like; and organic acids such as acetic acid, maleic acid, tartaric acid, salicylic acid, citric acid, benzoic acid, pamoic acid, sulfonic acid, and the like. Exemplary bases include inorganic bases and organic bases. Salts derived from inorganic bases are those well-known to a person skilled in the art, including but not limited to ammonium, sodium, potassium, calcium, magnesium, and the like. Salts derived composition of the present application is from about 0.0005% to about 30% based on the total amount of the composition (w/w). In some embodiments, the amount of the active ingredient in the composition of the present application is from about 0.0005% to about 10% based on the total amount of the composition (w/w). In some embodiments, the amount of the active ingredient in the composition of the present application is from about 0.0005% to about 5% based on the total amount of the composition (w/w).

The amphipathic molecule of the present application may be any molecule having both a hydrophilic group and a hydrophobic group. The amphipathic molecule includes surfactants and other materials which have surface activity, such as short chain fatty acids or fatty alcohols.

In some preferred embodiments, the amphipathic molecule used in the present application may be a surfactant.

The surfactant used in the present application may be an ionic surfactant or a non-ionic surfactant conventionally used in the pharmaceutics.

The ionic surfactant includes anionic surfactants, cationic surfactants and amphipathic surfactants.

In some embodiments of the present application, for ionic surfactants, those having low water-solubility are preferred.

Exemplary ionic surfactants include but not limited to anionic surfactants such as salts of fatty acids, sulfated compounds, sulfonated compounds, and the like; cationic surfactants such as quaternary ammonium compounds, and the like; and amphipathic surfactants such as amino acids, betaines, and the like.

Exemplary non-ionic surfactants include but not limited to polyethylene glycols such as fatty alcohol-polyoxyethylene ether (AEO), alkylphenol ethoxylates, fatty acid ethoxylates, polyoxyethylene fatty amine, ethylene xoide-propylene oxide block copolymerized ethers, and the like; polyols such as monoalcohol esters, ethylene glycol esters, glycerol esters, neopentyl-type polyol esters, sorbitol esters, sorbitan esters, glycosyl esters, alkyl glucosides, and the like; nitrogen-containing non-ionic surfactants such as alkyl alcohol amides, amine oxides, and the like; and sterol-derived non-ionic surfactants.

In some embodiments, the surfactant used in the present application may be a phospholipid. The phospholipid used in the present application is selected from natural phospholipids, including but not limited to phosphatidic acids, phosphatidyl glycerol (PG), cardiolipin, phosphatidyl choline, phosphatidyl ethanolamine, phosphatidyl serine (PS), phosphatidyl inositol (PI), plasmalogens, ether lipids, phosphatidyl ethanolamine (PE), soybean phosphatidyl choline (SPC) or eggyolk phosphatidyl choline (EPC), phosphatidic acid (PA), sphingomyelin (SPH), galactocerebroside, glucocerebroside, sulfatide, ganglioside, and the like; synthetic phospholipids, including but not limited to dipalmitoyl phosphatidyl choline (DPPC), distearoyl phosphatidyl choline (DSPC), distearoyl phosphatidyl ethanolamine (DSPE), hydrogenated soybean phosphatidyl choline (HSPC), PEGylated distearoyl phosphatidyl ethanolamine (DSPE-PEG), and the like. In some preferred embodiments, the phospholipids is eggyolk phosphatidyl choline (EPC) or hydrogenated soybean phosphatidyl choline (HSPC).

In some embodiments, the surfactant used in the present application may be cholesterols. In some preferred embodiments, the surfactant used in the present application may be cholesterol.

The amphipathic molecule added into the composition of the present application may be a mixture formed by combining one or more of the above surfactants.

In some embodiments, the surfactant used in the present application may also be a mixture of eggyolk phosphatidyl choline (EPC) and cholesterol.

The selection of a specific amphipathic molecule in the composition depends on various factors, such as the type, polarity and pH of the active molecule, the type and concentration of other additives existing in the composition, and the like. However, a person skilled in the art is able to perform the selection according to specific conditions of the composition. The selection and amount of the specific amphipathic molecule are based on forming a lipid-drug complex particulate.

The amount of the specific amphipathic molecule is normally from about 0.0001% to about 30.0% based on the total amount of the composition (weight percentage, w/w). In some embodiments, the amount of the specific amphipathic molecule is front about 0.005% to about 20% based on the total amount of the composition (w/w). In some embodiments, the amount of the specific amphipathic molecule is from about 0.005% to about 10% based on the total amount of the composition (w/w).

In addition to the amphipathic molecule, an organic acid and/or a salt thereof which is hardly soluble in water is also added into the sustained-release pharmaceutical composition of the present application. Therefore, the sustained-release performance is significantly improved. Although not verified by any theory, it is presumed that, in one hand, the active ingredient interacts with the organic acid and/or a salt thereof which is hardly soluble in water through electrostatic force, hydrophobic interaction, and coordination bonding to improve the lipophilicity and stability of the active ingredient and delay the release of the drug; in the other hand, adding an organic acid and/or a salt thereof which is hardly soluble in water into the composition would facilitate the dispersing of the formed lipid-drug complex in the oily solvent.

In some embodiments, the organic acid and/or a salt thereof which is hardly soluble in water is preferably that which is in the form of a solid under pharmaceutical conditions. In some embodiments, salts of organic acids are preferred.

The term “hardly soluble in water” as used herein refers to that the solubility of the organic acid or the salt thereof in 100 g water is less than or equal to 1 g.

In some embodiments, the organic acid and/or a salt thereof which is hardly soluble in water used in the composition of the present application may be selected from aliphatic acids or aromatic acids.

Exemplary organic acids include by not limited to saturated or unsaturated aliphatic acids having more than 10 carbon atoms, such as lauric acid, myristic acid, palmitic acid, oleic acid, linoleic acid, linolenic acid, stearic acid, palmitic acid, arachidonic acid, and the like. Exemplary aromatic acids include pamoic acid.

A salt of the organic acid which is hardly soluble in water may be selected from any salt of an organic acid which is hardly soluble in water, including but not limited to a salt of calcium, magnesium, barium, manganese, iron, copper, zinc, and aluminum, or may be a salt formed from any other organic acid, provided that it is hardly soluble in water and must be pharmaceutically acceptable (non-toxic).

In the composition of the present application, the organic acid which is hardly soluble in water, the salt of the organic acid which is hardly soluble in water, or a mixture thereof may be used.

In some embodiments, the organic acid and/or a salt thereof which is hardly soluble in water may be a combination of one or more of the above.

The specific amount of the organic acid and/or a salt thereof which is hardly soluble in water is normally from about 0.0001% to about 30% based on the total amount of the composition (weight percentage, w/w). In some embodiments, the specific amount of the organic acid and/or a salt thereof which is hardly soluble in water is from about 0.005% to about 20% based on the total amount of the composition (w/w). In some embodiments, the specific amount of the organic acid and/or a salt thereof which is hardly soluble in water is from about 0.005% to about 10% based on the total amount of the composition (w/w).

The sustained-release pharmaceutical composition of the present application may further comprise a pharmaceutically acceptable vehicle or excipient. Preferably, the vehicle or excipient is an oily solvent.

The oily solvent of the composition of the present application may be that conventionally used in the pharmaceutical field which is well-known to the person skilled in the art. Exemplary oily solvents include but not limited to natural plant oils such as soybean oil, Camellia oil, sesame oil, garlic oil, walnut oil, olive oil, corn oil, peanut oil, coconut oil, cottonseed oil, castor oil, and the like; refined plant oils; long-chain or medium-chain fatty acid glyceride; isopropyl myristate; ethyl linoleate; polyoxyethylene triolein; white oil; benzyl benzoate, and the like.

In some embodiments, the oily solvent may be a combination of one or more of the above.

In some preferred embodiments, the oily solvent may be soybean oil, or long-chain or medium-chain fatty acid glyceride.

The amount of the oily solvent is not strict, which may be selected by a person skilled in the art according to specific dosage form. The amount of the oily solvent is normally about 5% to about 99% of the total weight of the composition (weight percentage, w/w). In some embodiments, the amount of the oily solvent is about 30% to about 99% of the total weight of the composition (weight percentage, w/w). In some embodiments, the amount of the oily solvent is about 60% to about 99% of the total weight of the composition (weight percentage, w/w).

In some embodiments, the sustained-release formulation of the present application may further comprise a thickener. The thickener which may be used in the present application includes polymers such as PCL, PLGA, PLA, and the like. The amount of the thickener is from about 0.05% to about 10%, preferably about 0.5% to about 3.0%, based on the total weight of the sustained-release formulation (w/w).

In some embodiments, the sustained-release formulation of the present application may further comprise an antioxidant to ensure the stability of the injectable oil. The antioxidant which may be used in the present application may be selected from the group consisting of VE (vitamin E), BHT (butylated hydroxy toluene) BHA (butyl hydroxy anisd) and a mixture thereof. The amount of the antioxidant is from about 0.01% to about 2.0% (w/w), preferably about 0.05% to about 1.0%, based on the total weight of the sustained-release formulation (w/w).

A person skilled in the art would appreciate that the type and amount of the active ingredient, the amphipathic molecule, the organic acid and/or a salt thereof which is hardly soluble in water, and the oily solvent of the composition of the present application may be optionally combined according to the above ranges, provided that such a combination is able to achieve the object of the present application.

In some embodiments of the present application, the sustained-release pharmaceutical composition comprises from about 1 μg to about 500 mg of a peptide or protein thug, from about 1 μg to about 300 mg of a surfactant, from about 1 μg to about 300 in of a saturated or unsaturated aliphatic acid having more than 10 carbon atoms, and about 1 g of a natural plant oil.

In some embodiments of the present application, the sustained-release pharmaceutical composition comprises from about 5 μg to about 300 mg of a pharmaceutically acceptable salt of a peptide or protein drug, from about 50 μg to about 200 mg of a surfactant, from about 50 μg to about 200 mg of a salt which is hardly soluble in water of a saturated or unsaturated aliphatic acid having more than 10 carbon atoms, and about 1 g of a long-chain or medium-chain fatty acid glyceride.

In some embodiments of the present application, the sustained-release pharmaceutical composition comprises from about 5 μg to about 100 mg of a peptide or protein drug, from about 50 μg to about 100 mg of phospholipids-type surfactant, from about 50 μg to about 100 mg of a saturated or unsaturated aliphatic acid having more than 10 carbon atoms, and about 1 g of a long-chain or medium-chain fatty acid glyceride.

In some embodiments of the present application, the sustained-release pharmaceutical composition comprises from about 5 μg to about 50 mg of a nucleic acid drug, from about 1 μg to about 300 mg of a phospholipids-type surfactant, from about 1 μg to about 300 mg of an aromatic acid which is hardly soluble in water, and about 1 g of a long-chain or medium-chain fatty acid glyceride.

In some embodiments of the present application, the sustained-release pharmaceutical composition comprises from about 1 μg to about 500 mg of a saccharide or a non-peptide non-nucleic acid organic drug, from about 50 μg to about 200 mg of a phospholipids-type surfactant, from about 50 μg to about 200 mg of a salt of a saturated or unsaturated aliphatic acids having more than 10 carbon atoms, and about 1 g of a natural plant oil.

In some embodiments of the present application, the sustained-release pharmaceutical composition comprises from about 1 μg to about 500 mg of salmon calcitonin, from about 1 μg to about 200 mg of a natural phospholipids, from about 1 μg to about 50 mg of cholesterol, from about 1 μg to about 300 mg of a salt which is hardly soluble in water of a saturated or unsaturated aliphatic acid having more than 10 carbon atoms, and about 1 g of an oily solvent.

In some embodiments of the present application, the sustained-release pharmaceutical composition comprises from about 1 μg to about 500 mg of exenatide, from about 1 μg to about 200 mg of a natural phospholipids, from about 1 μg to about 50 mg of cholesterol, from about 1 μg to about 300 mg of a salt which is hardly soluble in water of a saturated or unsaturated aliphatic acid having more than 10 carbon atoms, and about 1 g of an oily solvent.

In some embodiments of the present application, the sustained-release pharmaceutical composition comprises from about 1 μg to about 500 mg of insulin, from about 1 μg to about 200 mg of a natural phospholipids, from about 1 μg to about 50 mg of cholesterol, from about 1 μg to about 300 mg of a salt which is hardly soluble in water of a saturated or unsaturated aliphatic acid having more than 10 carbon atoms, and about 1 g of an oily solvent.

In another aspect, the present application relates to an injectable sustained-release pharmaceutical formulation prepared from a sustained-release pharmaceutical composition disclosed above. The sustained-release pharmaceutical formulation may be administered through any route which is considered appropriate by a person skilled in the art. Preferably, the sustained-release pharmaceutical formulation is an injectable sustained-release pharmaceutical formulation. A person skilled in the art would appreciate that when the sustained-release pharmaceutical formulation is administered through injection, the components in the formulation shall be injectable components.

In a further aspect, the present application provides a process for preparing a sustained-release pharmaceutical formulation, comprising:

(1) dissolving or suspending an active ingredient into an aqueous solvent;

(2) dissolving or suspending an amphipathic molecule and an organic acid and/or a salt thereof which is hardly soluble in water into an organic solvent;

(3) dispersing the aqueous mixture of the active ingredient obtained in step (1) into the organic mixture obtained in step (2);

(4) removing the organic solvent from the mixture obtained in step (3);

(5) drying the products obtained in step (4) to form a solid; and

(6) dissolving or suspending the solid obtained in step (5) into an oily solvent.

A person skilled in the art would appreciate that steps (1) and (2) in the above process may not necessarily performed in the indicated sequence.

The aqueous solvent used in step (1) includes but is not limited to water, 0.9% sodium chloride aqueous solution, and any pharmaceutically suitable aqueous buffer. In some preferred embodiments, injectable water is used as an aqueous solvent. In some preferred embodiments. PBB buffer is used as an aqueous solvent.

The organic solvent used in step (2) may be selected from any organic solvent which has good solubilizing effects on the amphipathic molecule and the organic acid and/or salt thereof which is hardly soluble in water, and has low boiling point to enable it to be removed easily. Examples of the above organic solvents include but are not limited to dichloromethane, chloroform, ethyl ether, ethanol, methanol, n-propanol, iso-propanol, n-butanol, tert-butanol, acetone, acetonitrile, ethyl acetate. Different solvents may be selected according to the structure of the amphipathic molecule and the organic acid and/or a salt thereof which is hardly soluble in water used. The selection of the solvent is well-known to a person skilled in the art. In some preferred embodiments, dichloromethane is used as an organic solvent.

In step (3), in the preparation of a lipid-drug complex particulate, an active drug may be completely encapsulated in the lipid-drug complex particulate through processes such as ultrasonic dispersion method, reverse evaporation technique, film dispersion method, injection method, MVL preparation method, pH-gradient method, ammonium sulfate gradient method, or second encapsulation method, according to the nature of the active ingredient. In this step, it is important to uniformly mix and disperse the aqueous solution and the organic solution. In some preferred embodiments, ultrasonic dispersion method is employed.

In step (3), the operation temperature is selected according to the type of the amphipathic molecule used, the boiling point of the organic solvent used. Normally, the preparation process is carried out under a temperature in the range from −40° C. to 45° C. In some embodiments where HSPC is used as the amphipathic molecule, the process may be carried out under a temperature in the range from 40° C. to 45° C.

In step (4), the organic solvent is removed preferably through evaporation under reduced pressure to prevent degradation of the active ingredient in the formulation.

In some preferred embodiments, an appropriate amount of water may be added to the solid formed after removing the solvent to disperse the solid to obtain a uniform suspension, before the drying in step (5) is performed.

The drying process in step (5) may be lyophilization, spray drying or any other suitable drying process. The composition after drying is in the form of a solid.

In the lyophilization, a cryoprotectant is normally used to reduce the damage to the lipid-drug complex particulate during the freezing and melting process and to the leaking of the drug during the lyophilization. The effect of the cryoprotectant is to reduce the breaking of the bi-molecule layer membrane during the lyophilization, and to enable the lyophilized lipid particulate encapsulating the drug to be readily dispersed in the oily media. However, in the technical solution of the present application, the salt of the organic acid which is hardly soluble in water may play a role of cryoprotectant in addition of the role as disclosed above. Therefore, in some embodiments of the present application, there is no need to add any further cryoprotectant.

The solid obtain in the above step (5) is dissolved or dispersed in an oily solvent to form a solution or a suspension.

In the preparation process above, the sustained-release pharmaceutical formulation is preferably an injectable sustained-release formulation.

The present application may be used for biological drugs, or may be used for any hydrophilic injectable drugs such as small molecule compounds. The present application is particularly suitable for drugs such as peptides, proteins, nucleic acids and saccharides which have high polarity, good water-solubility and are unstable in water. Sustained-release formulations of various drugs such as peptides, proteins, nucleic acids were prepared herein with this technique and these formulations show sustained-release effect of 3 to 7 days in vitro. This type of the sustained-release pharmaceutical formulation may be preferably administered through intramuscular injection or subcutaneous injection, and keep releasing the active ingredient for 3 to 7 days.

The present application is further illustrated with the examples below. It shall be appreciated that these examples do not constitute any limitation to the scope of the present application.

### EXAMPLES

### Materials and Reagents

### Active Ingredients

Leuprorelin acetate: synthesized in the inventor's laboratory following a previously disclosed process (J. A. Vilchez-Martinez, et al. Biochem. Biophys. Res. Commun. 1974. 59:1226), HPLC purity >98%;

Naltrexone hydrochloride: presented by Wellso Parmaceutical Co. Ltd. China;

Thymopentin: synthesized in the inventor's laboratory following a previously disclosed process (G. Goldstein, et al. Science 1979, 204:1309). HPLC purity >98%;

Bovine serum albumins: purchased from Sigma, USA;

D33: DNA fragment containing 33 base pairs; 5′-d(TGC TCT CCA GGC TAG CTA CAA CGA CCT GCA CCT)-3′, synthesized in the inventor's laboratory following a previously disclosed process (Naruhisa Ota, et al. Nucleic Acid Research, 1998, 26(4):3385), HPLC purity >98%; all the base pairs used in the synthesis of D33 were purchased from Proligo LLC;

Exenatide: synthesized in the inventor's laboratory following a previously disclosed process (U.S. Pat. No. 6,528,486), HPLC purity >98%;

Pramlintide: synthesized in the inventor's laboratory following a previously disclosed process (U.S. Pat. No. 5,998,367), HPLC purity >98%;

Triptorelin acetate: synthesized in the inventor's laboratory following a previously disclosed process (D. H. Coy, et al. J Med. Chem. 1976, 19:423), HPLC purity >98%;

Somatostatin: synthesized in the inventor's laboratory following a previously disclosed process (A. M. Felix, et al. Int. J. Peptide Protein Res. 1980, 15:342), HPLC purity >98%;

ω-Interferon: presented by Southwest Pharmaceutical Co., Ltd., China;

Octreotide: synthesized in the inventor's laboratory following a previously disclosed process (W. Bauer, et al. Life Sci. 1982, 31:1133), HPLC purity >98%;

Salmon calcitonin: synthesized in the inventors laboratory following a previously disclosed process (U.S. Pat. No. 3,926,938), HPLC purity >98%;

Insulin: purchased from Tonghua Dongbao Pharmaceutical Co. Ltd., China;

### Amphiphilic Molecules

Eggyolk phosphatidyl choline (EPC), hydrogenated soybean phosphatidyl choline (HSPC), cholesterol: all purchased from Shanghai Toshisun Enterprise Co. Ltd. China;

Span 85: purchased from Fisher, USA;

### Aliphatic Acids and Salts Thereof

Aluminum stearate: purchased from Shanghai Bangcheng Chemical Co. Ltd., China;

Stearic acid: purchased from Beijing Shunyi Lisui Chemical Plant, China;

Oleic acid: purchased from Beijing Jinlong Chemical Reagents Co. Ltd., China;

Zinc stearate: purchased from Tianjin Langhu Chemical Engineering Co. Ltd., China;

### Oily Solvents

Injectable medium-chain oil, injectable soybean oil, both purchased from Tieling Beiya Pharmaceutical Oil Co. Ltd., China;

### Other Reagents

Ethyl ether: purchased from Tianjin Third Chemical Reagents Factory, China;

Methanol, dichloromethane: purchased from Beijing Chemical Plant, China;

PBS buffer: formulated following the appendix of Chinese Pharmacopoeia 2005;

Injectable water: purchased from Beijing Yahua Pharmaceutical Co. Ltd., China.

### Determination of In Vitro Accumulated Release

An active ingredient control was added into water to prepare standard active ingredient solutions with concentrations of 10 μg/mL, 20 μg/mL, 30 μg/mL, 50 μg/mL, 100 μg/mL, and 200 μg/mL. Absorbance A was determined for each solution with Folin-Ciocalteu method. The absorbance A was normalized with concentration to establish a standard curve regression equation.

An appropriate amount of the prepared sustained-release pharmaceutical formulation was placed into a 50 mL conical beaker equipped with a stopper, into which 10 mL of pH 7.10 phosphate buffer was added. The conical beaker was shaken in a rocking bed under a constant temperature of 37±1° C. and the shaking frequency was 70 r/m. At different time point, fixed amount of 200 μL of the sample was taken, and 200 μL of pH 7.10 phosphate buffer was added to make up the volume. The sample was centrifuged under 12,000 r/m for 10 mins. The supernatant fluid was taken as a sample solution. The absorbance of the sample solution was determined with the same method as described above. The concentration of the active ingredient was calculated with the regression equation obtained above.

The calculated accumulated amount of the drug was compared with the total amount of the added drug to calculate the percentage of accumulated release of the drug.

### Example 1

### Preparation and Sustained-Release Effects of Injectable Sustained-Release Formulation of Leuprorelin Acetate

1 mg of leuprorelin acetate was dissolved in 5 mL of 10 mmol/L PBS buffer (pH 7.0) as an aqueous phase. 20 mg of eggyolk phosphatidyl choline (EPC), 5 mg of cholesterol and 20 mg of aluminium stearate were dissolved in 20 mL of ethyl ether-methanol (10:1) mixed solvent as an organic phase. The above aqueous phase was dropwise added into the above organic phase at 30° C. under sufficient stirring. The resultant mixture was then treated in a water bath ultrasonic unit until a uniform emulsion system was formed. The mixture was evaporated under reduced pressure to remove the organic solvents and an appropriate amount of water was added to uniformly disperse the solid. The obtained suspension was lyophilized to remove water. 1 g of injectable medium-chain oil was added into the obtained solid product and stirred to disperse uniformly.

Following the above method for determining the in vitro accumulated release, the results of the in vitro accumulated release of the prepared sustained-release formulation of leuprorelin acetate in 7 days were determined and listed below:

| 1 day | 3 days | 5 days | 7 days |
|---|---|---|---|
| 20.6% | 37.4% | 76.0% | 94.0% |

### Example 2

### Preparation and Sustained-Release Effects of Injectable Sustained-Release Formulation of Naltrexone Hydrochloride

2 mg of naltrexone hydrochloride was dissolved in 5 mL of injectable water as an aqueous phase. 20 mg of hydrogenated soybean phosphatidyl choline (HSPC). 5 mg of cholesterol and 20 mg of aluminium stearate were dissolved in 20 mL of dichloromethane as an organic phase. The above aqueous phase was dropwise added into the above organic phase at 44° C. under sufficient stirring. The resultant mixture was then treated in a water bath ultrasonic unit until a uniform emulsion system was formed. The mixture was evaporated under the reduced pressure to remove the organic solvent and the obtained suspension was lyophilized to remove water. 1 g of injectable medium-chain oil was added into the obtained solid product and stirred to disperse uniformly.

Following the above method for determining the in vitro accumulated release, the results of the in vitro accumulated release of the prepared sustained-release formulation of naltrexone hydrochloride in 7 days were determined and listed below:

| 1 day | 3 days | 5 days | 7 days |
|---|---|---|---|
| 35.9% | 56.4% | 78.2% | 96.3% |

### Example 3

### Preparation and Sustained-Release Effects of Injectable Sustained-Release Formulation of Oligonucleotide

2 mg of D33 was dissolved in 5 mL of injectable water as an aqueous phase. 20 mg of EPC, 5 mg of cholesterol and 20 mg of aluminium stearate were dissolved in 20 mL of dichloromethane as an organic phase. The above aqueous phase was dropwise added into the above organic phase at 30° C. under sufficient stirring. The resultant mixture was then treated in a water bath ultrasonic unit until a uniform emulsion system was formed. The mixture was evaporated under the reduced pressure to remove the organic solvent and the obtained suspension was lyophilized to remove water. 1 g of injectable medium-chain oil was added into the obtained solid product and stirred to disperse uniformly.

Following the above method for determining the in vitro accumulated release, the results of the in vitro accumulated release of the prepared sustained-release formulation of oligonucleotide in 7 days were determined and listed below:

| 1 day | 3 days | 5 days | 7 days |
|---|---|---|---|
| 38.8% | 48.2% | 54.1% | 64.7% |

### Example 4

Following the same procedure as in Example 1, using different amphipathic molecules, different organic acids or salts which are hardly soluble in water, and different conditions of preparation, sustained-release formulations of different active ingredients were prepared, as shown in Table 1. The results of the in vitro accumulated release in 7 days were determined and listed in Table 2.

TABLE 1
Sustained-release formulations of different active ingredients

| | | | Organic acids or | | | | |
|---|---|---|---|---|---|---|---|
| | | | salts which are | | | Aqueous | |
| | Active | Amphipathic | hardly soluble in | Organic solvents | Aqueous phase | phase/organic | Oily solvents |
| | ingredients and | molecules and | water and amounts | and amounts | solvent and | phase mixing | and amounts |
| Nos. | amounts thereof | amounts thereof | thereof | thereof | amounts thereof | temperature (° C.) | thereof |
| 1 | Leuprorelin | EPC 20 mg | Stearic acid | Ethyl ether | Injectable water | 20 | Injectable |
| | acetate 2 mg | Cholesterol 5 mg | 5 mg | 20 mL | 5 mL | | soybean |
| | | | | | | | oil 1 g |
| 2 | Leuprorelin | EPC 20 mg | Oleic acid | Dichloromethane | 10 mmol/L pH | 30 | Injectable |
| | acetate 2 mg | Cholesterol 5 mg | 5 mg | 20 mL | 7.0 PBS buffer | | soybean |
| | | | | | 5 mL | | oil 1 g |
| 3 | Thymopentin | HSPC 20 mg | Zinc stearate | Dichloromethane | 10 mmol/L pH | 44 | Injectable |
| | 1 mg | Cholesterol 5 mg | 20 mg | 20 mL | 7.0 PBS buffer | | medium-chain |
| | | | | | 5 mL | | oil 1 g |
| 4 | Bovine serum | EPC 20 mg | Aluminium stearate | Dichloromethane | Injectable water | 30 | Injectable |
| | albumin 1 mg | Cholesterol 5 mg | 25 mg | 20 mL | 5 mL | | medium-chain |
| | | | | | | | oil 1 g |
| 5 | Leuprorelin | Span 85 20 mg | Aluminium stearate | Ethyl ether | 10 mmol/L pH | 20 | Injectable |
| | acetate 1 mg | Cholesterol 5 mg | 25 mg | 20 mL | 7.0 PBS buffer | | soybean |
| | | | | | 5 mL | | oil 1 g |
| 6 | Exenatide | EPC 20 mg | Zinc stearate | Dichloromethane | Injectable water | 30 | Injectable |
| | 2 mg | Cholesterol 5 mg | 20 mg | 20 mL | 5 mL | | medium-chain |
| | | | | | | | oil 1 g |
| 7 | Pramlintide | EPC 20 mg | Aluminium stearate | Dichloromethane | Injectable water | 30 | Injectable |
| | 2 mg | Cholesterol 5 mg | 20 mg | 20 mL | 5 mL | | medium-chain |
| | | | | | | | oil 1 g |
| 8 | Triptorelin | EPC 20 mg | Aluminium stearate | Dichloromethane | Injectable water | 30 | Injectable |
| | acetate 2 mg | Cholesterol 5 mg | 20 mg | 20 mL | 5 mL | | medium-chain |
| | | | | | | | oil 1 g |
| 9 | Somatostatin | EPC 20 mg | Aluminium stearate | Dichloromethane | Injectable water | 30 | Injectable |
| | 2 mg | Cholesterol 5 mg | 20 mg | 20 mL | 5 mL | | medium-chain |
| | | | | | | | oil 1 g |
| 10 | ω-Interferons | EPC 20 mg | Aluminium stearate | Dichloromethane | Injectable water | 30 | Injectable |
| | 2 mg | Cholesterol 5 mg | 20 mg | 20 mL | 5 mL | | medium-chain |
| | | | | | | | oil 1 g |
| 11 | Octreotide | EPC 20 mg | Aluminium stearate | Dichloromethane | Injectable water | 30 | Injectable |
| | 5 mg | Cholesterol 5 mg | 20 mg | 20 mL | 5 mL | | medium-chain |
| | | | | | | | oil 1 g |
| 12 | Salmon | EPC 20 mg | Aluminium stearate | Dichloromethane | Injectable water | 30 | Injectable |
| | calcitonin 1 mg | Cholesterol 5 mg | 20 mg | 20 mL | 5 mL | | medium-chain |
| | | | | | | | oil 1 g |
| 13 | Insulin | EPC 20 mg | Aluminium stearate | Dichloromethane | Injectable water | 30 | Injectable |
| | 1 mg | Cholesterol 5 mg | 20 mg | 20 mL | 5 mL | | medium-chain |
| | | | | | | | oil 1 g |

TABLE 2
In vitro release results of different sustained-release
pharmaceutical formulations

Accumulated release of drugs %

| Nos. | 1 day | 3 days | 5 days | 7 days |
|---|---|---|---|---|
| 1 | 32.1 | 77.2 | 96.7 | — |
| 2 | 40.3 | 83.3 | 100 | — |
| 3 | 41.2 | 68.1 | 91.0 | — |
| 4 | 24.0 | 50.5 | 65.9 | 87.1 |
| 5 | 44.2 | 71.0 | 91.2 | — |
| 6 | 21.0 | 47.1 | 58.0 | 62.7 |
| 7 | 21.7 | 60.2 | 71.3 | 94.2 |
| 8 | 29.6 | 52.9 | 79.9 | 93.3 |
| 9 | 28.4 | 58.2 | 68.0 | 82.0 |
| 10 | 13.9 | 21.2 | 45.4 | 68.2 |
| 11 | 27.0 | 46.8 | 71.2 | 81.8 |
| 12 | 37.5 | 47.5 | 62.7 | 86.7 |
| 13 | 32.1 | 56.6 | 80.1 | 97.2 |

The data in Table 2 indicated that the sustained-release pharmaceutical formulations prepared with the process of the present application have a good sustained-release effect for various types of active ingredients.

### Example 5

Following the same procedure as in Example 1, using leuprorelin acetate as an active ingredient, and using different amounts of aluminium stearate, sustained-release pharmaceutical formulations were prepared and the in vitro accumulated release thereof were determined and listed in Tables 3 and 4.

TABLE 3
Sustained-release formulations of leuprorelin acetate

| | Amount of | Amount of |
|---|---|---|
| Nos. | leuprorelin acetate | aluminium stearate |
| 1 | 2 mg | — |
| 2 | 1 mg | 20 mg |
| 3 | 1 mg | 50 mg |

TABLE 4
Results of the in vitro accumulated release of the sustained-release
formulations of leuprorelin acetate

Accumulated release
of drugs %

| Nos. | 1 d | 3 d | 5 d | 7 d |
|---|---|---|---|---|
| 1 | 53.4 | 69.7 | 84.1 | 99.8 |
| 2 | 20.6 | 37.4 | 76.0 | 94.0 |
| 3 | 17.2 | 41.6 | 76.7 | 100.0 |

Adding a small amount of aluminium stearate in the formulations would improve the release performance of the drug, but the release performance of the drug would reduce when an amount of aluminium stearate is too high.

### Example 6

The effects of the preparation manner on the in vitro sustained-release performance of leuprorelin acetate were investigated.

Without the preparation procedure, an amphipathic molecule (e.g. EPC, Span 85) was directly added into an oily solvent, followed by adding an active drug, dispersed uniformly, and the in vitro accumulated release of the drug was determined with the method described above. The results demonstrated that more than 90% of the active drugs were released within 1 day. The sustained-release performance was significantly lower than that of the formulation obtained through the preparation process of the present application. The results were shown in Table 5.

TABLE 5

| | | Amphipathic | | |
|---|---|---|---|---|
| | Leuprorelin acetate | molecules and amount | | Accumulated |
| Nos. | and amount thereof | thereof | Preparation manner | release % |
| 1 | 2.1 mg | EPC 20 mg | Direct dissolving | 98.9 |
| | | Cholesterol 5 mg | without preparation | (24 h) |
| | | | procedure | |
| 2 | 2.2 mg | EPC 20 mg | with preparation | 53.4 |
| | | Cholesterol 5 mg | procedure | (24 h) |
| 3 | 1.8 mg | Span 85 100 mg | Direct dissolving | 100 |
| | | Cholesterol 5 mg | without preparation | (12 h) |
| | | | procedure | |
| 4 | 2.1 mg | Span 85 100 mg | with preparation | 36.2 |
| | | Cholesterol 5 mg | procedure | (24 h) |

The results demonstrated that the formulations obtained through the preparation process of the present application showed better stability and better sustained-release performance.

### Example 7

The effects of sustained-release formulations of calcitonin on bone mineral density in ovariectomized rats were investigated.

The animals used were wiste female rats weighed 160-200 g. The instrument used was Bone Densitometer (LUNAR) manufactured by General Electric Company, USA.

Experimental procedure: the rats were anesthetized by intramuscular injection of saiantong anaesthesia compound formula (10 mg/kg), shaved at the abdominal region, and cut alone the middle line of the lower abdomen. Ovaries at both sides of the rats were dissociated and excised. The muscles and skins at the abdominal region were then sutured. Penicillin was intramuscularly injected after the operation at 2 times per day for 3 days. On day 3 after the operation, formulation 12 as prepared in Example 4 was administered once by subcutaneous injection at dosage of 8 μg/kg. The bone mineral densities in the lumbar vertebra in the third and forth week after the administration were determined, respectively, and were compared with the pseudo-operation group in which the ovaries were not excised from the rats and the model group in which the ovaries were excised from the rats but no calcitonin formulation was injected.

The results demonstrated that comparing with the model group, the bone mineral densities in the lumbar vertebra of the rats significantly increased in the third and Forth week after the administration (p<0.05).

TABLE 6
Effects of sustained-release formulations of calcitonin on
bone mineral density in ovariectomized rats

| | | bone mineral densities | |
|---|---|---|---|
| | | in lumbar vertebra after | |
| | Dosage | administration (g/cm²) | |
| Groups | (μg/kg) | Third week | Fourth week |
| Pseudo-operation group | — | 0.225 ± 0.157 | 0.232 ± 0.112 |
| Model group | — | 0.195 ± 0.011⁼ | 0.197 ± 0.013⁼⁼ |
| Sustained-release | 8.0 | 0.211 ± 0.011* | 0.213 ± 0.018* |
| formulation of calcitonin | | | |

Notes:
⁼p < 0.05,
⁼⁼p < 0.01, comparing with the pseudo-operation group:
*p < 0.05, comparing with the model group;
n = 8

### Example 8

The effects of sustained-release formulations of exenatide on blood glucose levels of mice were investigated.

Following similar procedure as in Example 1, using exenatide as an active ingredient, injectable water as an aqueous phase solvent, dichloromethane as an organic phase solvent, and using the components as listed in Table 7, sustained-release formulations of exenatide were prepared.

TABLE 7
Sustained-release formulations of exenatide

| | | Eggyolk | | | Injectable |
|---|---|---|---|---|---|
| | | phosphatidyl | | Aluminium | medium-chain |
| Nos. | Exenatide | choline | Cholesterol | stearate | oil |
| Formulation 1 | 5 mg | 20 mg | 10 mg | — | 1 g |
| Formulation 2 | 5 mg | 20 mg | 10 mg | 20 mg | 1 g |
| Formulation 3 | 5 mg | 20 mg | 10 mg | 50 mg | 1 g |

The animals used were male KK-Ay mice, aged 8-10 weeks and raised under the conditions which complied with corresponding standards.

### Grouping and Drug Treatment of Animals

KK-Ay mice were randomly grouped into solvent control group, positive control group, formulation 1 group, formulation 2 group, and formulation 3 group according to weights and blood glucose values, with 5 animals in each group.

Solvent control group: injectable medium-chain oil was administered by intramuscular injection with a single dosage of 100 μl per animal, 50 μl at each hind leg;

Positive control group: a solution of exenatide in PBS buffer was administered subcutaneously at the neck region with a dosage of 0.06 μg/100 μl at 5:30 pm each day;

Formulation 1 group, formulation 2 group, and formulation 3 group: formulations 1, 2 and 3 in the above table were administered by intramuscular injection, respectively, at a single dosage of 100 μl per animal, 50 μl at each hind leg.

Changes in the blood glucose level were monitored at 8:30-9:00 am each day. On the eighth day after administration, an additional administration was performed; 4 μg/100 μl was administered to each animal in formulation 1 group, formulation 2 group, and formulation 3 group; and 0.6 μg/100 μl was administered to each animal in positive control group. Starting from the ninth day, 0.18 μg/100 μl was administered to each animal in positive control group twice a day.

The blood glucose level's of KK-Ay mice after starving for 4 h on 17th to 41st day after administration (Table 8) and after starving for 12 h on 35th day after administration (Table 9) were determined. The experiment results showed that all of the formulations 1, 2 and 3 demonstrated significant and sustained effects of reducing blood glucose in KK-Ay mice after starving for 4 h or 12 h, which may last for 24 to 27 days after administration, in which the effects of formulation 2 are more significant.

TABLE 8
Blood glucose levels of KK-Ay mice after starving for 4 h on 17th to 41st day
after administration

| | Blood Glucose (mmol/L) | | | | |
|---|---|---|---|---|---|
| Time | Solvent control | Positive control | Formulation 1 | Formulation 2 | Formulation 3 |
| (day) | group | group | group | group | group |
| 17 | 14.88 ± 4.10 | 11.5 ± 3.47 | 10.98 ± 3.15 | 10.72 ± 2.24 | 11.64 ± 3.04 |
| 22 | 23.56 ± 2.50 | 16.18 ± 3.98 | 18.72 ± 4.28 | 16.92 ± 3.03 | 18.04 ± 3.96* |
| 27 | 19.88 ± 3.36 | 14.06 ± 3.58 | 16.90 ± 5.03 | 14.54 ± 3.53 | 16.28 ± 3.27 |
| 32 | 17.54 ± 3.51 | 12.56 ± 2.68 | 14.60 ± 2.65 | 15.14 ± 0.87 | 15.40 ± 6.07 |

TABLE 9
Blood glucose levels of KK-Ay mice after starving for
12 h on 35th day after administration

| | Blood Glucose |
|---|---|
| Groups | (mmol/L) |
| Solvent control group | 10.3 ± 2.23 |
| Positive control group | 6.16 ± 1.59** |
| Formulation 1 group | 6.58 ± 1.13* |
| Formulation 2 Group | 6.84 ± 1.26* |
| Formulation 3 group | 7.34 ± 1.10* |

*p < 0.05,
**p < 0.01, comparing with control group

In addition, formulations 2 and 3 showed significant effects of suppressing food intake on the first day of administration (specific to this drug). During the whole process of the experiment, formulation 2 showed effects of suppressing food intake which are similar to the positive control drug.

### Example 9

In vitro release and in vivo efficacy of sustained-release formulations of insulin were investigated.

Following similar procedure as in Example 1, using the amounts of solvents as listed in Table 10, sustained-release formulations of insulin were prepared from 5.0 mg of insulin, 20 mg of eggyolk phosphatidyl choline, 10 mg of cholesterol, 20 mg of aluminium stearate and 1 g of injectable medium-chain oil, and the in vitro accumulated release of the formulations was determined (Table 11).

TABLE 10
Sustained-release formulations of insulin

| | Aqueous phase solvents and amounts | Organic solvents and amounts |
|---|---|---|
| Formulations | thereof | thereof |
| 1 | Aqueous solution of acetic acid 5 mL | Dichloromethane 20 mL |
| 2 | Aqueous solution of acetic acid 5 mL | Dichloromethane 10 mL |
| 3 | Aqueous solution of acetic acid 0.5 mL | Dichloromethane 20 mL |
| 4 | Aqueous solution of acetic acid 0.5 mL | Dichloromethane 9.5 mL |
| | | Acetone 0.5 mL |
| 5 | Aqueous solution of acetic acid 5 mL | Dichloromethane 19.5 mL |
| | | Tert-butanol 0.5 ml |
| 6 | Aqueous solution of acetic acid 5 mL | Dichloromethane 19.5 mL |
| | | Ethanol 0.5 mL |
| 7 | Aqueous solution of acetic acid 5 mL | Dichloromethane 19.5 mL |
| | | Isopropanol 0.5 mL |
| 8 | Aqueous solution of acetic acid 5 mL | Dichloromethane 9.5 mL |
| | | Tert-butanol 1.0 mL |

TABLE 11
In vitro accumulated release of sustained-release
formulations or insulin

Accumulated release of drug %

| Formulations | 1 day | 3 days | 5 days | 9 days |
|---|---|---|---|---|
| 1 | 3.75 | 10.0 | 40.1 | 57.3 |
| 2 | 13.2 | 27.2 | 47.9 | 100 |
| 3 | 6.31 | 17.6 | 69.3 | 97.5 |
| 4 | 24.1 | 33.8 | 55.8 | 92.3 |
| 5 | 4.66 | 14.1 | 31.8 | 45.9 |
| 6 | 19.6 | 26.2 | 64.1 | 100 |
| 7 | 21.1 | 46.7 | 77.0 | 100 |
| 8 | 18.0 | 32.3 | 76.5 | 96.3 |

The data in Table 11 demonstrated that the testing formulations slowly released the drug in vitro in at least 9 days.

### In Vivo Efficacy of Sustained-Release Formulations of Insulin

Experimental procedure: basically following the previously disclosed process (Lijiang Song, et al., Observation of effect of a glucose-reducing medical care capsule on model mice, Journal of Chinese Medicine Research, 6(1):53-55, 2006). After starved and fed by only water for 24 hrs. mice were intraperitoneally injected with 160 mg/kg of streptozotocin. After 72 hrs, blood glucose levels at fasting for 6 h were determined. Mice with blood glucose of 15-30 mmol/L were classified as qualified for the model and were evenly divided into different groups. On the fourth day, mice were injected with the insulin formulations 1-8, respectively, while the control group was only given the auxiliaries. After injection, blood glucose levels were determined at fasting for 6 hrs at 3 h, 24 h, 3 d, 5 d, 7 d and 9 d, respectively. One drop of blood was taken from each animal by cutting tails thereof, and was added onto a OneTouch® Basic® Blood Glucose Meter glucose oxidase Test Strip manufactured by LifeScan Inc. of Johnson & Johnson Ltd. to measure blood glucose levels. The results were listed in Tables 12-13 below.

TABLE 12
Efficacy of sustained-release formulations of insulin
in treating mice having streptozotocin-induced diabetes

| | | Blood glucose (mmol/L) | | | | | |
|---|---|---|---|---|---|---|---|
| | Dosage | | 3 hrs after | | | | |
| Groups | (U/kg) | 0 day | administration | 1 day | 5 days | 9 days | 14 days |
| Control | | 26.6 ± 2.1 | 26.3 ± 2.0 | 21.8 ± 5.7 | 19.3 ± 3.9 | 20.0 ± 3.5 | 21.6 ± 3.9 |
| Formulation | 10 | 26.5 ± 1.8 | 22.6 ± 2.4 | 7.1 ± 4.8* | 11.8 ± 4.1* | 12.2 ± 1.1* | 22.1 ± 2.2 |
| 1 | 30 | 27.5 ± 1.8 | 21.1 ± 2.2 | 3.0 ± 1.1* | 9.6 ± 5.4* | 12.6 ± 0.5* | 19.2 ± 2.3 |

*P < 0.05

It can be seed from the results listed in Table 12 that for insulin formulation 1 which was subcutaneous injected at 10 U/kg and 30 U/kg, the peak of the effects of reducing blood glucose is at 24 hrs, and the effects lasted for 9-14 days.

TABLE 13
Efficacy of different sustained-release formulations of insulin
in treating mice having streptozotocin-induced diabetes

| | Dosage | Blood glucose (mmol/L) | | | | | |
|---|---|---|---|---|---|---|---|
| Groups | (U/kg) | 0 day | 1 day | 3 days | 5 days | 7 days | 9 days |
| Control | | 19.1 ± 2.5 | 18.3 ± 4.4 | 22.4 ± 4.3 | 20.6 ± 4.2 | 21.9 ± 3.1 | 22.7 ± 4.2 |
| group | | | | | | | |
| Formulation | 10 | 19.1 ± 2.5 | 5.5 ± 1.0* | 7.1 ± 4.8* | 8.3 ± 1.2* | 11.8 ± 4.1* | 22.7 ± 4.2 |
| 5 | 30 | 19.1 ± 2.5 | 2.6 ± 0.6* | 3.0 ± 1.1* | 6.1 ± 4.0* | 9.6 ± 5.4* | 21.5 ± 3.2 |
| | 60 | 19.0 ± 2.4 | 1.9 ± 0.8* | 2.4 ± 1.0* | 6.4 ± 6.2* | 13.5 ± 5.5* | 16.4 ± 6.4 |
| Formulation | 10 | 21.4 ± 3.5 | 8.0 ± 4.1* | 15.4 ± 5.0* | 21.5 ± 2.6 | 21.2 ± 1.6 | 24.0 ± 3.2 |
| 2 | 30 | 20.3 ± 1.9 | 2.8 ± 0.9* | 3.3 ± 1.5* | 18.3 ± 8.8 | 17.8 ± 6.1 | 22.4 ± 4.7 |
| | 60 | 20.5 ± 2.1 | 3.1 ± 2.4* | 9.1 ± 8.2* | 22.0 ± 0.8 | 20.9 ± 0.6 | 21.6 ± 3.0 |
| Formulation | 30 | 22.4 ± 1.2 | 5.7 ± 2.7* | 19.0 ± 3.03 | 22.8 ± 1.6 | 19.7 ± 2.7 | 22.8 ± 3.9 |
| 3 | | | | | | | |

*P < 0.05. comparing with control group

It can be seen from the results listed in Table 13 that the significant effects of formulation 3 in reducing glucose lasted for 1 day, the significant effects of formulation 2 in reducing glucose lasted for 3 days, and the significant effects of formulation 5 in reducing glucose lasted for 7 days.

A person skilled in the art would understand that the term “such as” or “for example” as used herein represents “including, but not limited to”.

Although the present application is described through the above embodiments and specific descriptions, but the present applicant is not limited thereto. In view of the disclosure of the present application, a person skilled in the art may make modifications or changes the technical features in the above embodiments without departure from the spirit of the present application, and these modifications or changes are within the scope of the present application.

1. A sustained-release pharmaceutical composition, comprising a therapeutically effective amount of an active ingredient, an amphipathic molecule, an organic acid and/or a salt thereof which is hardly soluble in water, and an oily solvent.

2. The sustained-release pharmaceutical composition according to claim 1, wherein the active ingredient is a hydrophilic drug.

3. The sustained-release pharmaceutical composition according to claim 2, wherein the hydrophilic drug is selected from the group consisting of peptides or proteins; nucleic acids; saccharides or non-peptide non-nucleic acid organic drugs; and a mixture thereof.

4. The sustained-release pharmaceutical composition according to claim 3, wherein the peptides or proteins are selected from pituitary polypeptides such as adrenal cortical hormone, gastrin, vasopressin, oxytocin, melanoma stimulating hormone, and the like; gastrointestinal peptides such as secretin, gastrin, cholecystokinin, gastrone, vasoactive intestinal peptide, pancreatic polypeptide, neurotensin, frog skin peptide, and the like; hypothalamic peptides such as thyrotropin releasing hormone, gonadotropin releasing hormone, somatostatin, growth hormone releasing hormone, MSH cytokine inhibiting hormone, and the like; brain peptides such as enkephalin, neoendorphine, endorphin, memory peptide, and the like; kinins such as angiotensins I, II, III, and the like; glutathione; calcitonin; sleep-inducing peptides; pineal peptides; solcoseryl; thymosin; thymopentin; octreotide; exenatide; pramlintide; fibrous proteins; fibrinogens; gastric mucin; gelatin; gelatin sponge; protamines; endostatins; exendin; parotin; hirudin; hepatocyte growth factors; leuprorelin; triptorelin; nafarelin; goserelin; buserelin; bovine serum albumins; insulin; erythropoietin (EPO); tumor necrosis factors; vaccines; auxins; glucagons; serum albumins; gamma-globulins; trypsin inhibitors; erythropoietins; interferons; interleukins; colony-stimulating factors (GM-CSFs); luteinizing hormones, phytohemagglutinin, trichosanthin, plant toxic proteins; and antibodies.

5. The sustained-release pharmaceutical composition according to claim 3, wherein the nucleic acids include DNA fragments such as DNA fragment comprising 33 base pair, chemically modified DNA fragments such as thio-DNA fragments, RNA fragments, chemically modified RNA fragments, polyinosinic acid, mecapto polycytidylic acid, cAMP, CTP, CDP-choline, GMP, IMP, AMP, inosine, UTP, NAD, NADP, 2-methylmercapto furan inosinic acid, bisformyl cAMP, 6-mercaptopurine, 6-mercaptopurinenucleoside, 6-thiopurine, 5-fluorouracil, furan fluorouracil, 2-deoxynucleoside, cytarabine hydrochloride, and antiviral enzyme plasmid gene.

6. The sustained-release pharmaceutical composition according to claim 3, wherein the saccharides or non-peptide non-nucleic acid organic drugs are selected from polysaccharide drugs such as heparin, pilose antler polysaccharides, polysaccharide from stichopus japonicus, chitosan, dextran, lentinan, tremella polysaccharide, pachymaran, ganoderma lucidum polysaccharides, and the like; chemically synthesized drugs such as naltrexone hydrochloride, morphine hydrochloride mitoxantrone hydrochloride, cortisone acetate, and the like.

7. The sustained-release pharmaceutical composition according to any one of claims 1-6, wherein the amount of the active ingredient is from about 0.0001% to about 50% (weight percentage, w/w), particularly from about 0.0005% to about 30% (w/w), particularly from about 0.0005% to about 10% (w/w), particularly from about 0.0005% to about 5% (w/w), based on the total amount of the composition.

8. The sustained-release pharmaceutical composition according to claim 1, wherein the amphipathic molecule is a surfactant.

9. The sustained-release pharmaceutical composition according to claim 8, wherein the surfactant is a non-ionic surfactant.

10. The sustained-release pharmaceutical composition according to claim 9, wherein the non-ionic surfactant is selected from polyethylene glycols such as fatty alcohol-polyoxyethylene ether (AEO), alkylphenol ethoxylates, fatty acid ethoxylates, polyoxyethylene fatty amine, ethylene xoide-propylene oxide block copolymerized ethers, and the like; polyols such as monoalcohol esters, ethylene glycol esters, glycerol esters, neopentyl-type polyol esters, sorbitol esters, sorbitan esters, glycosyl esters, alkyl glucosides, and the like; nitrogen-containing non-ionic surfactants such as alkyl alcohol amides, amine oxides, and the like; and sterol-derived non-ionic surfactants.

11. The sustained-release pharmaceutical composition according to claim 8, wherein the surfactant is a phospholipid.

12. The sustained-release pharmaceutical composition according to claim 11, wherein the phospholipid is selected from natural phospholipids, including but not limited to phosphatidic acids, phosphatidyl glycerol (PG), cardiolipin, phosphatidyl choline, phosphatidyl ethanolamine, phosphatidyl serine (PS), phosphatidyl inositol (PI), plasmalogens, ether lipids, phosphatidyl ethanolamine (PE), soybean phosphatidyl choline (SPC) or eggyolk phosphatidyl choline (EPC), phosphatidic acid (PA), sphingomyelin (SPH), galactocerebroside, glucocerebroside, sulfatide, ganglioside, and the like; synthetic phospholipids, including but not limited to dipalmitoyl phosphatidyl choline (DPPC), distearoyl phosphatidyl choline (DSPC), distearoyl phosphatidyl ethanolamine (DSPE), hydrogenated soybean phosphatidyl choline (HSPC), PEGylated distearoyl phosphatidyl ethanolamine (DSPE-PEG), and the like.

13. The sustained-release pharmaceutical composition according to claim 12, wherein the phospholipid is selected from eggyolk phosphatidyl choline (EPC) or hydrogenated soybean phosphatidyl choline (HSPC).

14. The sustained-release pharmaceutical composition according to claim 8, wherein the surfactant is a cholesterol.

15. The sustained-release pharmaceutical composition according to claim 8, wherein the surfactant is any mixture of a non-ionic surfactant, a phospholipid and a cholesterol.

16. The sustained-release pharmaceutical composition according to claim 1, wherein the amount of the amphipathic molecule is from about 0.0001% to about 30.0% (weight percentage, w/w), particularly from about 0.005% to about 20% (w/w), particularly from about 0.005% to about 10% (w/w), based on the total amount of the composition.

17. The sustained-release pharmaceutical composition according to claim 1, wherein the organic acid and/or a salt thereof which is hardly soluble in water is selected from lauric acid, myristic acid, palmitic acid, oleic acid, linoleic acid, linolenic acid, stearic acid, palmitic acid, arachidonic acid, pamoic acid and/or a salt thereof.

18. The sustained-release pharmaceutical composition according to claim 17, wherein the salt of the organic acid which is hardly soluble in water is selected from a salt of calcium, magnesium, barium, manganese, iron, copper, zinc, and aluminum of the organic acid which is hardly soluble in water.

19. The sustained-release pharmaceutical composition according to claim 1, wherein the amount of the organic acid and/or a salt thereof which is hardly soluble in water is from about 0.0001% to about 30% (weight percentage, w/w), from about 0.005% to about 20% (w/w), from about 0.005% to about 10% (w/w), based on the total amount of the composition.

20. The sustained-release pharmaceutical composition according to claim 1, wherein the oily solvent is selected from the group consisting of injectable natural plant oils, refined plant oils, long-chain or medium-chain fatty acid glycerides, benzyl benzoate, and a mixture thereof, and is preferably selected from injectable soybean, or a long-chain or medium-chain fatty acid glycerides.

21. A sustained-release pharmaceutical formulation, comprising a sustained-release pharmaceutical composition according to claim 1.

22. The sustained-release pharmaceutical formulation according to claim 21, being an injectable sustained-release pharmaceutical formulation.

23. A process for preparing a sustained-release pharmaceutical formulation of claim 21, comprising:
(1) dissolving or suspending an active ingredient into an aqueous solvent;
(2) dissolving or suspending an amphipathic molecule and an organic acid and/or a salt thereof which is hardly soluble in water into an organic solvent;
(3) dispersing the aqueous mixture of the active ingredient obtained in step (1) into the organic mixture obtained in step (2);
(4) removing the organic solvent from the mixture obtained in step (3);
(5) drying the product obtained in step (4) to form a solid; and
(6) dissolving or suspending the solid obtained in step (5) into an oily solvent.

24. The process according to claim 23, wherein an appropriate amount of water is added to the solid formed after removing the solvent in step (4) to disperse the solid to obtain a uniform suspension.

25. The process according to claim 23, wherein the drying process in step (5) is lyophilization.

26. The process according to claim 23, wherein the sustained-release pharmaceutical formulation is an injectable sustained-release pharmaceutical formulation.

27. A process for treating diseases in a subject, comprising administrating to the subject a therapeutically effective amount of a pharmaceutical composition of claim 1 or a sustained-release pharmaceutical formulation of claim 21.
